(19) United States
(12) Patent Application Publication
(10) Pub. No.: US 2026/0155234 A1
(43) Pub. Date: Jun. 4, 2026

(54) SYSTEM AND METHOD FOR REMOTELY MANAGING MENTAL HEALTH OF A USER

(71) Applicant: Chelsey Campbell, Tamarac, FL (US)

(72) Inventor: Chelsey Campbell, Tamarac, FL (US)

(21) Appl. No.: 19/402,168

(22) Filed: Nov. 26, 2025

Related U.S. Application Data
(60) Provisional application No. 63/726,360, filed on Nov. 29, 2024.

(51) Int. Cl. G16H 20/70

(52) U.S. Cl. CPC G16H 20/70

(57) ABSTRACT

A system for remotely managing mental health of a user includes a tag, a user device, and a server. The user device receives a uniform resource locator (URL) from the tag and renders a web resource for providing digital mental health resources corresponding to the URL. The server receives user input data associated with the mental health of the user from the user device and determines the digital mental health resources for the user. The server further monitors interactions of the user with the web resource and the digital mental health resources to generate behavioural data and resource usage data, and dynamically updates, using machine learning models, the digital mental health resources provided to the user based on identified unlabelled clusters, patterns, or relationships in the user input data, the behavioural data, and the resource usage data.

### CROSS-REFERENCE TO RELATED APPLICATIONS

The present application claims priority under 35 U.S.C. § 119(e) to U.S. Provisional Patent Application No. 63/726360, titled “SYSTEM AND METHOD FOR PROVIDING MENTAL HEALTH RESOURCES” filed Nov. 29, 2024, the disclosure of which is herein incorporated by reference in its entirety.

### BACKGROUND OF THE INVENTION

Mental health management has emerged as a critical aspect of overall well-being, with an increasing prevalence of mental health disorders globally. It influences how people think, feel, and behave in their lives, as well as how they manage stress, build relations, and make decisions. Prioritizing mental health management is essential as it enables individuals to handle challenges, maintain resilience, and live fulfilling lives. Traditional approaches involve face-to-face therapy sessions, which may not be possible in every situation due to geographical constraints, personal stigma, financial cost, time consuming process, and various other factors.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 is an exemplary system for remotely managing mental health of a user, in accordance with some embodiments.

FIG. 2 is a block diagram of an exemplary user device for use within the system of FIG. 1, in accordance with some embodiments.

FIG. 3 is a block diagram of an exemplary server for use within the system of FIG. 1, in accordance with some embodiments.

FIGS. 4A and 4B illustrate a flow diagram of a method for remotely managing the mental health of the user, in accordance with some embodiments.

FIG. 5 is a block diagram of an optimization cycle for optimizing digital mental health resources provided by the server of FIG. 3, in accordance with some embodiments.

Skilled artisans will appreciate that the elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

In an aspect, a system for remotely managing mental health of a user is described. The system includes an object, a tag adhered to the object, a user device, and a server communicatively coupled to the user device. The tag includes a memory for storing a uniform resource locator (URL) associated with a web resource for providing digital mental health resources. The user device is configured to receive the URL from the tag when the user device is positioned within a predefined distance from the tag, render the web resource corresponding to the URL on a user device interface of the user device, and obtain, via the user device interface, user input data associated with the mental health of the user through one or more graphical elements of the web resource. The server is associated with the web resource and is configured to receive, by a server transceiver, the user input data associated with the mental health of the user and, determine, by a server processor, mental health state of the user based on the user input data. The server is further configured to determine, by the server processor, digital mental health resources corresponding to the mental health state of the user using one or more machine learning models and provide, by the server transceiver, the digital mental health resources to the user through the user device interface. Further, the server is configured to monitor, by the server processor, interactions of the user with the web resource and the digital mental health resources provided through the user device interface to generate behavioural data and resource usage data, respectively, and provide, by the server processor, the user input data, the behavioural data, and the resource usage data to the one or more machine learning models to identify unlabelled clusters, patterns, or relationships in the user input data, the behavioural data, and the resource usage data associated with the user. Further, the server is configured to dynamically update, by the server processor, the digital mental health resources provided to the user through the user device interface based on the identified clusters, patterns, or relationships using the one or more machine learning models and obtain, by the server processor, user feedback on the digital mental health resources provided to the user through the user device interface. The server is further configured to train, by the server processor, the one or more machine learning models based on the user feedback using a reinforcement learning technique and dynamically update, by the server processor, the digital mental health resources provided to the user through the user device interface based on the user feedback using the one or more machine learning models. Further, the server is configured to continuously monitor, by the server processor, the web resource to obtain updated data associated with one or more of the user input data, the behavioural data, the resource usage data, and the user feedback, and repeat, by the server processor, the step associated with updating the digital mental health resources using the one or more machine learning models upon receiving the updated data. The user device is further configured to display, on the user device interface, the digital mental health resources provided by the server.

In another aspect, a method for remotely managing mental health of a user is described. The method includes receiving, by a user device transceiver of a user device, a uniform resource locator (URL) from a tag when the user device is positioned within a predefined distance from the tag. The URL is associated with a web resource for providing digital mental health resources. The method further includes rendering, on a user device interface of the user device, the web resource corresponding to the URL, obtaining, via the user device interface, user input data associated with the mental health of the user through one or more graphical elements of the web resource, and receiving, by a server transceiver of a server, the user input data associated with the mental health of the user. Further, the method includes determining, by a server processor of the server, mental health state of the user based on the user input data, determining, by the server processor, digital mental health resources corresponding to the mental health state of the user using one or more machine learning models, and providing, by the server transceiver, the digital mental health resources to the user through the user device interface of the user device. The method further includes monitoring, by the server processor, interactions of the user with the web resource and the digital mental health resources provided through the user device interface of the user device to generate behavioural data and resource usage data, respectively, providing, by the server processor, the user input data, the behavioural data, and the resource usage data to the one or more machine learning models to identify unlabelled clusters, patterns, or relationships in the user input data, the behavioural data, and the resource usage data associated with the user, and dynamically updating, by the server processor, the digital mental health resources provided to the user through the user device interface based on the identified clusters, patterns, or relationships using the one or more machine learning models. The method further includes obtaining, by the server processor, user feedback on the digital mental health resources provided to the user through the user device interface, training, by the server processor, the one or more machine learning models based on the user feedback using a reinforcement learning technique, and dynamically updating, by the server processor, the digital mental health resources provided to the user through the user device interface based on the user feedback using the one or more machine learning models. Further, the method includes continuously monitoring, by the server processor, the web resource to obtain updated data associated with one or more of the user input data, the behavioural data, the resource usage data, and the user feedback, repeating, by the server processor, the step associated with updating the digital mental health resources using the one or more machine learning models upon receiving the updated data, and displaying, on the user device interface, the digital mental health resources provided by the server.

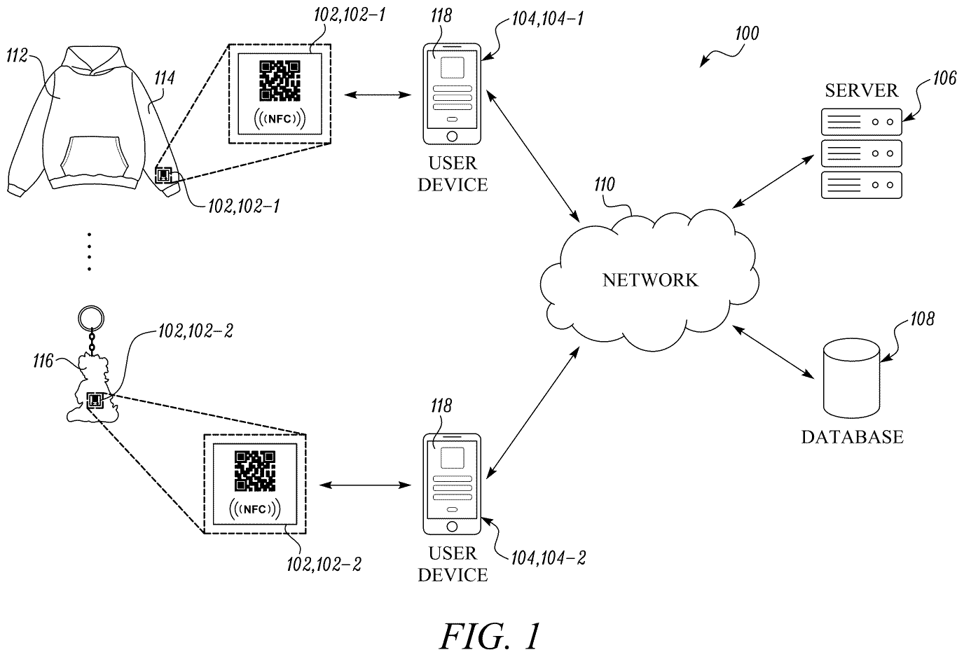

FIG. 1 illustrates a system 100 for remotely managing mental health of a user, in accordance with some embodiments. The mental health of a user corresponds to a state of emotional and psychological well-being of the user. In accordance with various embodiments, the system 100 is configured to remotely manage the mental health of the user by providing recommendations (for example, digital mental health resources) to a user device associated with the user. The digital mental health resources include services, tools, and technologies provided through digital platforms to help users maintain and improve their mental health. For example, the digital mental health resources include, but is not limited to, digital self-help resources, remote counseling with therapists, and any other digital resource now known or developed in future. The digital self-help resources include videos, websites, documents, books, podcasts, exercises, meditation support, online forums, access to support groups, monthly reports, interactive coping activities, text/chat support, chatbots, challenges, alerts, support helpline, and other similar types of resources provided through digital platforms. In accordance with various embodiments, the system 100 is configured to provide personalized recommendations (for example, personalized digital mental health resources) to the users based on one or more of user input data, resource usage data, behavioural data, and user feedback data, as will be described hereinafter.

The system 100 includes a plurality of objects 112, 116, a plurality of tags 102 (for example, but not limited to, tags 102-1 and 102-2), a plurality of user devices 104 (for example, but not limited to, user devices 104-1 and 104-2), a server 106, and a database 108. Each tag 102 communicates with its corresponding user device 104 using a short-range wireless technology, such as, Near Field Communication (NFC). Although FIG. 1 illustrates the short-range wireless technology to include NFC, it would be appreciated that the short-range wireless technology can correspond to any other short-range wireless technology, such as, Bluetooth, Radio Frequency Identification (RFID), Zigbee, and other such wireless technologies now known or developed in future. The user devices 104, the server 106, and the database 108 are communicatively coupled to each other via a network 110. The network 110 includes, but is not limited to, a wide area network (WAN) (for example, a transport control protocol/internet protocol (TCP/IP) based network), a cellular network, or a local area network (LAN) employing any of a variety of communications protocols as is now known or in the future developed.

Each tag 102 is configured to store a uniform resource locator (URL) associated with a web resource 118 (for example, a web portal, a web page, or any other suitable interface now known or in the future developed) for providing the digital mental health resources. In some embodiments, the web resource 118 corresponds to a portal or page accessed through a mobile application installed on the user device 104. For example, the tag 102 is an NFC tag, a radio frequency identification (RFID) tag, or any other tag capable of storing and transmitting the URL. Each tag 102 is configured to wirelessly receive power from its corresponding user device 104 and transmit the URL stored in its memory to the corresponding user device 104 when the user device 104 is positioned within a predefined distance from the tag 102. The predefined distance depends on a range of the short-range wireless technology utilized to enable transmission of the URL from the tag 102 to the user device 104. In some embodiments, the tag 102 stores multiple URLs corresponding to multiple web resources (not shown) for providing the digital mental health resources depending upon one or more inputs from the user Although not described in detail, a person skilled in the art would appreciate that the tag 102 also includes, in addition to the memory for storing the URL, an antenna, an integrated circuit chip, and various other components known to support the operations of the tag 102.

In accordance with various embodiments, the tags 102 are adhered to the objects 112, 116. The objects 112, 116 include wearables (for example, clothing, watches, belts, wallets, backpacks, or other such accessories), non-wearables (for example, key chains, epoxy resin-based models, umbrellas, phone cases), or any other item now known or in the future developed. In some embodiments, the objects 112, 116 are associated with a user. The tags 102 are adhered to the objects 112, 116 through adhesion, sewing, embedding, or any other adherence method now known or developed in the future. For example, as shown in FIG. 1, the tag 102-1 is sewn to a sleeve 114 of a clothing item 112 and the tag 102-2 is embedded into a key chain 116. By adhering the tag 102 to the object 112, 116, a user of the object 112, 116 easily accesses the digital mental health resources on his/her user device 104, for example, by tapping the tag 102 using the user device 104.

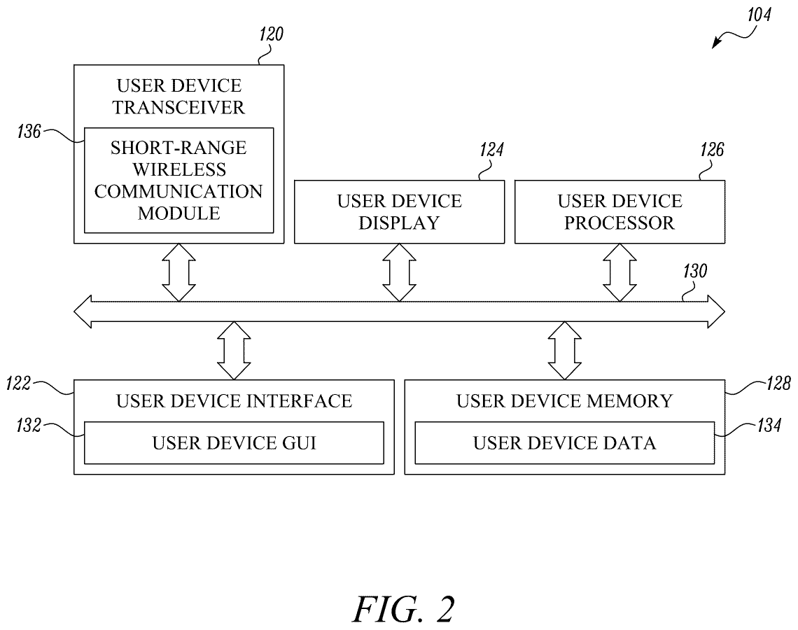

Each user device 104 is configured to receive the URL from the corresponding tag 102 when the user device 104 is positioned within the predefined distance from the tag 102. In some embodiments, when the tag 102 corresponds to the NFC tag, the user device 104 is configured to activate the corresponding tag 102 by generating a magnetic field (as is well known in the art) to receive the URL from the tag 102. The user device 104, upon receiving the URL, displays the web resource 118, for example, on a user device interface (described below), for providing the digital mental health resources to its user. Each user device 104 also operates as an interface for the corresponding user to interact with the server 106. The user device 104 is a mobile phone, an electronic tablet, or any other communication device now known or in the future developed for receiving the URL from the tag 102. The various components of the user device 104 will now be described hereinafter with respect to FIG. 2. It should be appreciated by those of ordinary skill in the art that FIG. 2 depicts the user device 104 in a simplified manner and a practical embodiment includes additional components and suitably configured logic to support known or conventional operating features that are not described in detail herein. Although the user device 104 is shown and described to be implemented within a single communication device, it is contemplated that the one or more components of the user device 104 are alternatively be implemented in a distributed computing environment.

Referring to FIG. 2, the user device 104 includes, among other components, a user device transceiver 120, a user device interface 122, a user device display 124, a user device processor 126, and a user device memory 128. The components of the user device 104, including the user device transceiver 120, the user device interface 122, the user device display 124, the user device processor 126, and the user device memory 128, cooperate with one another to enable operations of the user device 104. Each component communicates with one another via a user device local interface 130. The user device local interface 130 includes, but is not limited to, one or more buses or other wired or wireless connections, as is known in the art. The user device local interface 130 includes additional elements, which are omitted for simplicity, such as controllers, buffers (caches), drivers, repeaters, and receivers, among many others, to enable communications. Further, the user device local interface 130 includes address, control, and/or data connections to enable appropriate communications among the aforementioned components.

As illustrated, the user device 104 includes the user device transceiver 120 to transmit data associated with the user to the server 106 and/or the database 108 and receive one or more outputs (for example, the digital mental health resources) from the server 106. The user device transceiver 120 includes a transmitter circuitry and a receiver circuitry to enable the user device 104 to communicate with the server 106 and/or the database 108. In this regard, the transmitter circuitry includes appropriate circuitry to transmit one or more of the user input data, the resource usage data, the behavioural data, and the user feedback data to the server 106 and/or the database 108, and the receiver circuitry includes appropriate circuitry to receive the one or more outputs from the server 106.

It will be appreciated by those of ordinary skill in the art that the user device 104 includes a single user device transceiver 120 as shown, or, alternatively, separate transmitting and receiving components, for example, but not limited to, a transmitter, a transmitting antenna, a receiver, and a receiving antenna. In some embodiments, the user device transceiver 120 also includes a short-range wireless communication module 136 to receive the URL from the tag 102. Although not shown, the short-range wireless communication module 136 can include a short-range wireless communication reader integrated circuit and a short-range wireless communication antenna to communicate with the tag 102. It would be appreciated that the components and functionality of the short-range wireless communication reader integrated circuit and the short-range wireless communication antenna integrated in the user device 104 is well known in the art and is not described here for the sake of brevity. In some embodiments (not shown), the short-range wireless communication module 136 can be a separate unit from

### The User Device Transceiver 120.

In accordance with various embodiments, the user device interface 122 is configured to receive inputs from and/or provide the outputs to the user. The inputs are provided via a touch screen display (such as, the user device display 124), a camera, a touch pad, a keyboard, a microphone, a recorder, a mouse, or any other user input mechanism now known or developed in the future. The outputs are provided via a display device, such as the user device display 124, a speaker, a haptic output, or any other output mechanism now known or developed in the future. The user device interface 122 further includes a serial port, a parallel port, an infrared (IR) interface, a universal serial bus (USB) interface, and/or any other interface herein known or developed in the future.

In accordance with some embodiments, the user device interface 122 includes a user device graphical user interface (GUI) 132 through which the user communicates with the server 106. The user device GUI 132 corresponds to the web resource 118. As discussed above, the web resource 118 is the web page, the web portal or any other suitable interface. The user device GUI 132 is accessed through a web browser on the user device 104 or within a mobile application installed on the user device 104. The user device GUI 132 includes one or more of graphical elements including, but not limited to one or more of dialogue boxes, window, web forms, and/or the like. The graphical elements are used in conjunction with text to prompt the user for inputs or display the outputs to the user in response to one or more instructions from the server 106.

The user device display 124 is configured to display reports, dialogue boxes, web forms, data, images, videos, and the like. The user device display 124 includes a display screen or a computer monitor, and/or the like devices now known or in the future developed. In accordance with some embodiments, the user device display 124 is configured to display, on the user device GUI 132, the digital mental health resources provided by the server 106.

The user device memory 128 is a non-transitory memory configured to store a set of instructions that are executable by the user device processor 126 to perform predetermined operations. For example, the user device memory 128 includes any of the volatile memory elements (for example, random access memory (RAM)), non-volatile memory elements (for example, read only memory (ROM)), and combinations thereof. Moreover, the user device memory 128 incorporates electronic, magnetic, optical, and/or other types of storage media. In accordance with some embodiments, the user device memory 128 is also configured to store one or more of the user input data, the resource usage data, the behavioural data, the user feedback data, and the digital mental health resources. In some embodiments, the user device data 134 includes personal data associated with the user and the application associated with the user device GUI 132.

The user device processor 126 is configured to execute the instructions stored in the user device memory 128 to perform the predetermined operations. The user device processor 126 includes one or more microprocessors, microcontrollers, DSPs (digital signal processors), state machines, logic circuitry, or any other device or devices that process information or signals based on operational or programming instructions. The user device processor 126 is implemented using one or more controller technologies, such as Application Specific Integrated Circuit (ASIC), Reduced Instruction Set Computing (RISC) technology, Complex Instruction Set Computing (CISC) technology, or any other similar technology now known or in the future developed. The user device processor 126 is configured to cooperate with other components of the user device 104 to perform operations pursuant to communications and the one or more instructions from the server 106.

Referring back to FIG. 1, the database 108 stores one or more of the user input data, the resource usage data, the behavioural data, the user feedback data, and the digital mental health resources corresponding to each user device 104. In some embodiments, the database 108 is configured to receive one or more of the user input data, the resource usage data, the behavioural data, and the user feedback data from the user devices 104 via the network 110 and transmit the one or more of the user input data, the resource usage data, the behavioural data, the user feedback data, and the digital mental health resources to the server 106, upon receiving a request from the server 106.

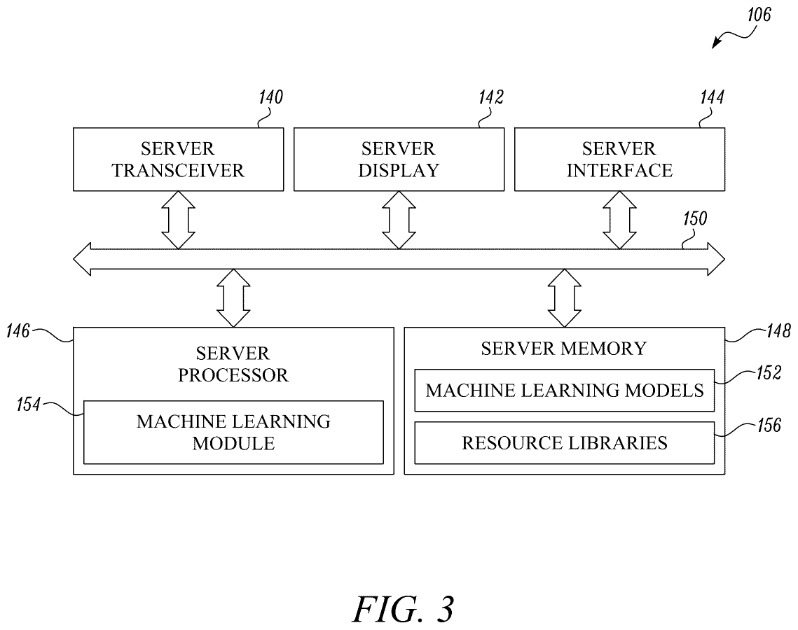

With continued reference to FIG. 1, the server 106 is configured to provide the output (for example, the digital mental health resources) on the user device 104 based on one or more of the user input data, the resource usage data, the behavioural data, and the user feedback data. As shown in FIG. 3, the server 106 includes a plurality of electrical and electronic components, providing power, operational control, communication, and the like functions, within the server 106. For example, the server 106 includes, among other components, a server transceiver 140, a server interface 144, a server display 142, a server processor 146, and a server memory 148. It should be appreciated by those of ordinary skill in the art that FIG. 3 depicts the server 106 in a simplified manner and a practical embodiment includes additional components and suitably configured logic to support known or conventional operating features that are not described in detail herein. It will further be appreciated by those of ordinary skill in the art that the server 106 is a personal computer, desktop computer, tablet, smartphone, or any other computing device now known or developed in the future.

Further, although the server 106 is shown and described to be implemented within a single computing device, it is contemplated that the one or more components of the server 106 are alternatively implemented in a distributed computing environment, without deviating from the scope of the claimed subject matter. It will further be appreciated by those of ordinary skill in the art that the server 106 alternatively functions within a remote server, cloud computing device, or any other remote computing mechanism now known or developed in the future. The server 106 is a cloud environment incorporating the operations of the server transceiver 140, the server display 142, the server interface 144, the server processor 146, and the server memory 148, and various other operating modules to serve as a software and/or as a service model for the user device 104. In an embodiment, the server 106 and the user device 104 are one computing device incorporating or performing the operations of all the components of the server 106 and the user device 104. In an embodiment, the functionalities of the server 106 and the user device 104 are distributed in two or more computing devices.

The components of the server 106, including the server transceiver 140, the server display 142, the server interface 144, the server processor 146, and the server memory 148, communicates with one another via a server local interface 150. The server local interface 150 includes, namely, but not limited to, one or more buses or other wired or wireless connections, as is known in the art. The server local interface 150 have additional elements, such as, but not limited to, controllers, buffers (caches), drivers, repeaters, and receivers, among many others, to enable communications. Further, the server local interface 150 includes address, control, and/or data connections to enable appropriate communications among the aforementioned components.

The server transceiver 140 includes a transmitter circuitry and a receiver circuitry (not shown) to enable the server 106 to communicate data to and acquire data from other devices, such as, the user device 104 and the database 108. In this regard, the transmitter circuitry includes appropriate circuitry to transmit data to the user device 104 and/or the database 108, and the receiver circuitry includes appropriate circuitry to acquire data from the user device 104 and/or the database 108. The transmitter circuitry and the receiver circuitry together form a wireless transceiver to enable wireless communication with the user device 104 and/or the database 108. It will be appreciated by those of ordinary skill in the art that the server 106 includes a single server transceiver 140 as shown, or alternatively separate transmitting and receiving components, for example, but not limited to, a transmitter, a transmitting antenna, a receiver, and a receiving antenna.

In some embodiments, the server interface 144 is configured to receive data from and/or provide output to an individual (for example, a programmer). The data is provided via a touch screen display (such as, the server display 142), a camera, a touch pad, a keyboard, a microphone, a recorder, a mouse, or any other user input mechanism now known or developed in the future. The output is provided via a display device, such as the server display 142, a speaker, a haptic output, or any other output mechanism now known or developed in the future. The server interface 144 further includes a serial port, a parallel port, an infrared (IR) interface, a universal serial bus (USB) interface, and/or any other suitable interface now known or developed in the future. The server display 142 includes a display screen or a computer monitor now known or in the future developed.

The server memory 148 is a non-transitory memory configured to store a set of instructions that are executable by the server processor 146 to perform the predetermined operations. For example, the server memory 148 includes any of the volatile memory elements (for example, random access memory (RAM)), non-volatile memory elements (for example read only memory (ROM)), and combinations thereof. The software in the server memory 148 includes one or more software programs, each of which includes an ordered listing of executable instructions for implementing logical functions. Moreover, the server memory 148 incorporates electronic, magnetic, optical, and/or other types of storage media.

The server memory 148 stores the user input data, the resource usage data, the behavioural data, the user feedback data, and the digital mental health resources associated with one or more users. The server memory 148 also includes one or more machine learning models 152 executed by a machine learning module 154. In some embodiments, the server memory 148 includes a first dataset and a second dataset for training the one or more machine learning models 152, as will be described hereinafter.

In some embodiments, the server memory 148 includes a plurality of resource libraries 156. Each resource library 156 is tailored to specific moods and life situations, offering a range of interactive, therapist approved resources, and is unlocked through the activation of the tag 102 on the corresponding object 112, 116. The resource library 156 includes book and podcast recommendations, original video content (for example, interviews, documentaries), guided visualizations and games, affirmation exercises, journaling prompts, educational reads, breathwork, meditation practices, and crisis resource directories. In some alternate embodiments, the server memory 148 includes links to the plurality of resource libraries 156, and historical and public mental health databases. The historical and public mental health databases include data on mental health awareness and emotional triggers. In some embodiments, the historical and public mental health databases also include widely accepted therapeutic techniques and coping mechanisms from psychology and behavioural sciences.

In some embodiments, the server memory 148 is located external to the server 106, such as, for example, an external hard drive connected to the server interface 144. In some embodiments (not shown), the server memory 148 is located external (for example, remotely) and connected to the server 106 through a network and accessed via the server interface 144. In some embodiments, the externally located server memory 148 corresponds to the database 108. Alternatively, in other embodiments, the server memory 148 and the database 108 are distinct independent storage units. In such cases, the above indicated data is stored either in the server memory 148 or the database 108 or in a distributed manner in both the server memory 148 and the database 108.

The server processor 146 is configured to execute the instructions stored in the server memory 148 to perform the predetermined operations, for example, the detailed functions of the server 106, as will be described hereinafter. The server processor 146 includes one or more microprocessors, microcontrollers, DSPs (digital signal processors), state machines, logic circuitry, or any other device or devices that process information or signals based on operational or programming instructions. The server processor 146 can be implemented using one or more controller technologies, such as Application Specific Integrated Circuit (ASIC), Reduced Instruction Set Computing (RISC) technology, Complex Instruction Set Computing (CISC) technology, or any other technology now known or in the future developed.

The server processor 146 includes a machine learning module 154 configured to learn and adapt itself to continuous improvement in changing environments. The machine learning module 154 employs any one or combination of the following computational techniques: neural network, constraint program, fuzzy logic, classification, conventional artificial intelligence, symbolic manipulation, fuzzy set theory, evolutionary computation, cybernetics, data mining, approximate reasoning, derivative-free optimization, decision trees, and/or soft computing. The machine learning module 154 implements an iterative learning process. The learning is based on a wide variety of learning rules or training algorithms. In an embodiment, the learning rules include one or more of back-propagation, patter-by-pattern learning, supervised learning, unsupervised learning, reinforcement learning, and/or interpolation. The machine learning module 154 is configured to implement one or more machine learning algorithms to train the machine learning models 152 for providing the digital mental health resources based on the one or more of the user input data, the resource usage data, the behavioural data, and the user feedback data received on the user device 104. In accordance with some embodiments of the present description, the machine learning algorithm utilizes any machine learning methodology, now known or in the future developed, for providing the digital mental health resources. For example, the machine learning methodology utilized includes one or a combination of: Linear Classifiers (Logistic Regression, Naive Bayes Classifier); Nearest Neighbor; Support Vector Machines; Decision Trees; Boosted Trees; Random Forest; and/or Neural Networks. The machine learning module 154 continually evolves the digital mental health resources in real time with new inputs from the user device 104. The machine learning intent is to continually revise the web resource 118 with the updated digital mental health resources over time based on updated data associated with one or more of the user input data, the resource usage data, the behavioural data, and the user feedback data. The functionalities and operations of the server processor 146, including the machine learning module 154, will be described hereinafter in greater detail.

The server processor 146 is configured to train the machine learning models 152 by a combination of a supervised, an unsupervised, and a reinforcement learning techniques. In accordance with various embodiments, the server processor 146 is configured to train the machine learning models 152 by obtaining the first training dataset that corresponds to a labelled dataset from existing historical and public mental health databases on mental health awareness/interventions and emotional triggers. The dataset includes widely accepted therapeutic techniques and coping mechanisms from psychology and behavioural sciences. These datasets are obtained from clinical studies, mental health research papers, and public datasets related to behavioural health of individuals. For example, the labelled dataset includes historical data of indicators associated with a plurality of mental health states and corresponding digital mental health resources to manage each mental health state of the plurality of health states. The mental health state corresponds to a condition of emotional, psychological, and social well-being of the user.

The supervised learning technique includes training the machine learning models 152 on the labelled dataset. In accordance with various embodiments, the server processor 146 is configured to train the machine learning models 152 by obtaining the first training dataset including historical data of indicators associated with a plurality of mental health states and corresponding digital mental health resources to manage each mental health state of the plurality of health states. The server processor 146 is configured to train, using the supervised learning technique, the machine learning models 152 on the first training dataset to (i) define one or more indicators corresponding to each mental health state of the plurality of health states, (ii) identify the mental health state of a user based on the one or more indicators, and (iii) determine digital mental health resources corresponding to each mental health state of the plurality of health states. For example, the supervised learning technique enables the machine learning models 152 to recognize indicators based on usage of words such as, “nervous”, “on edge”, “overwhelmed”, and the like words, and identify the mental health state of the user as “anxious” based on such indicators. The supervised learning technique also enables the machine learning models 152 to learn patterns of emotions and mental health symptoms (for example, recognizing common indicators of stress, anxiety, or depression) and match them to appropriate coping strategies or resources. For example, the machine learning models 152, when executed by the machine learning module 154, are trained to recognize how users describe feelings of anxiety (for example, “nervous”, “on edge”, “overwhelmed”) and learn to recommend specific digital mental health resources, such as, grounding techniques or deep breathing exercises when similar moods are logged.

The unsupervised learning technique includes training the machine learning models 152 on an unlabelled dataset to identify hidden patterns. In accordance with various embodiments, the server processor 146 is configured to obtain a second training dataset including historical user input data, historical behavioural data, and historical resource usage data of a plurality of users and train, using the unsupervised learning technique, the machine learning models 152 on the second training dataset to identify unlabelled clusters, patterns, or relationships in the user input data, the behavioural data, and the resource usage data of the plurality of users. In accordance with various embodiments, identifying unlabelled clusters, patterns, or relationships in the user input data, the behavioural data, and the resource usage data associated with the user includes identifying connections between different mental health states of the user. This enables the machine learning models 152 to discover clusters of emotional states or behaviour patterns that weren't explicitly labelled. This provides better understanding about how users'moods fluctuate over time, and identify subtle, non-obvious connections between different emotional states, without pre-defined outcomes. For example, the machine learning models 152 determine that the user who regularly tracks feelings of stress in the morning also tend to benefit from a particular morning routine. Accordingly, the machine learning models 152 provide suggestion even when the user hasn't explicitly requested for a suggestion.

The reinforcement learning technique includes training the machine learning models 152 to adjust recommendations (for example, the digital mental health resources) based on the user feedback received via the user device 104. This enables the machine learning models 152 to track how the user responds to its suggestions (for example, does the user interact with the suggested resource? or does the user rate the suggestion as helpful?). The machine learning models 152, upon receiving a positive reinforcement, (for example, when the user consistently follows the provided suggestions) learn and improve over time. This allows continuous, personalized improvement in the suggestions/recommendations provided by the machine learning models 152 based on real-time interactions. For example, when the user dismisses meditation suggestions but frequently uses breathing exercises, the machine learning models 152 learn to deprioritize meditation in favour of breathing-related resources.

In accordance with various embodiments, a process of training the machine learning model 152 includes an initial dataset preprocessing, a machine learning model training, a validation and testing, and a continuous learning. For example, the initial dataset preprocessing includes collection of mood logs, the behavioural data, and the user feedback which is cleaned and pre-processed. In some embodiments, the initial dataset preprocessing includes passing the language data (for example, journaling entries, notes entered by the user) through one or more natural language processing (NLP) models to detect emotional sentiment, stress-related keywords, and mood trends. For example, the training includes training the machine learning models 152 on historical data of emotional triggers and coping strategy effectiveness, using the supervised learning technique for known responses to emotions and the unsupervised learning technique for identifying new patterns. The reinforcement learning technique is used to refine suggestions based on the real-time user feedback.

In accordance with various embodiments, the validation and testing of the trained machine learning models 152 is performed based on the user feedback and interaction. The machine learning module 154 regularly validates the recommendations (for example, the digital mental health resources) provided by the machine learning models 152 with ongoing feedback loops from the user, adjusting for incorrect predictions or inappropriate recommendations. For example, the continuous learning provides the machine learning models 152 to remain adaptive, learning over time as more users engage with the system 100 and input more data. This refines recommendations to better suit individual preferences and behavioural patterns.

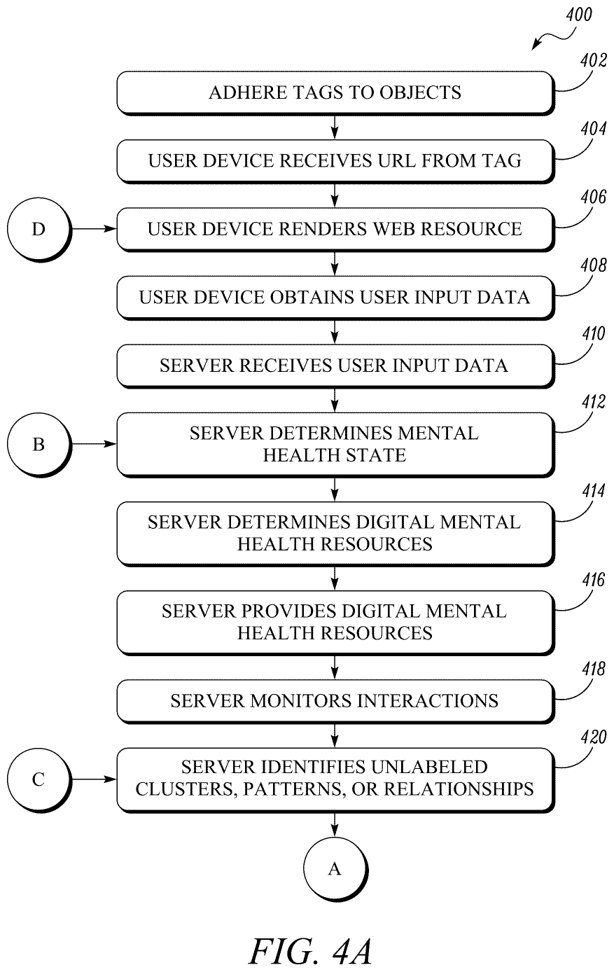

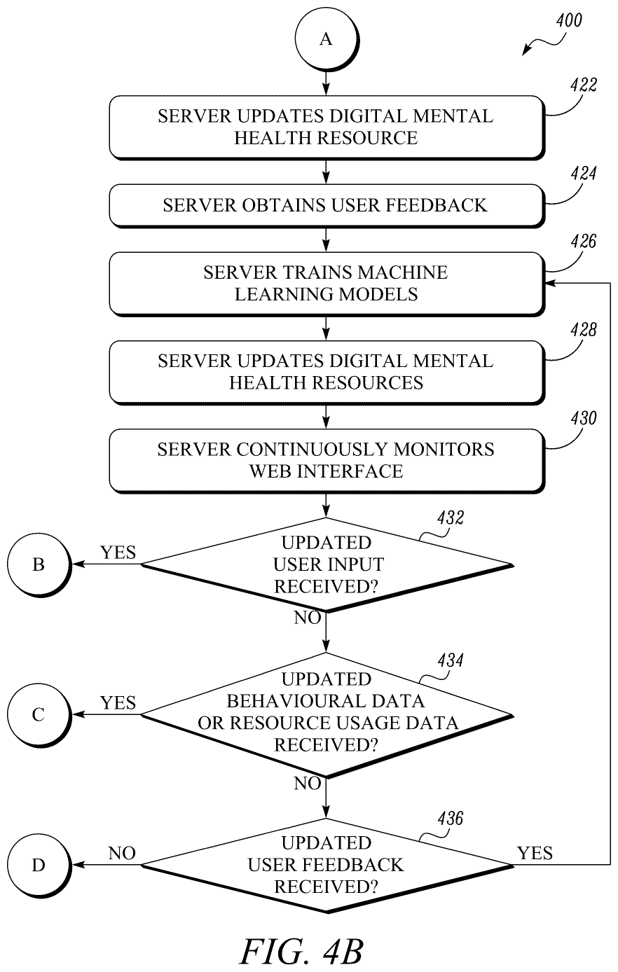

FIGS. 4A and 4B illustrates a flow diagram of a method for remotely managing the mental health of the user, in accordance with various embodiments. The method 400 begins at 402 by adhering the tags 102 to the objects 112 and 116. As discussed above, the tag 102-1 is sewn to the sleeve 114 of the clothing item 112 and the tag 102-2 is embedded into the key chain 116. At 404, at least one of the user devices 104 (for example, the user device 104-1) receives the URL from its corresponding tag 102 (for example, the tag 102-1) when the user device 104 is positioned within the predefined distance from the tag 102. When the tag 102 corresponds to the NFC tag, the user device 104 receives the URL by activating the NFC tag, for example, by tapping the tag 102 at a back of the user device 104.

At 406, the user device 104, upon receiving the URL, renders the web resource 118 corresponding to the URL on the user device interface 122. In accordance with various embodiments, the web resource 118 is provided by the server 106 and allows the user device 104 to access the digital mental health resources that are personalized based on inputs from the user device 104, as will be described in detail hereinafter.

At 408, the user device 104 obtains, via the user device interface 122, the user input data associated with the mental health of the user through one or more graphical elements of the web resource 118. In accordance with various embodiments, the user input data corresponds to mood and emotion data that explicitly defines an emotional state of the user. For example, the emotional state of the user corresponds to as ‘depressed, ‘anxious’ or similar predefined emotional states. The mood and emotion data corresponds to data self-reported by the user during mood tracking sessions. The mood tracking sessions are organized at a predetermined time interval (for example, daily or weekly) to enable the users to log their emotions/mood and potential emotional triggers. In some embodiments, the user device 104 receives the mood and emotion data from the user throughout the day. This enables the server 106 to provide a comprehensive and real-time overview of the user's mental health state to the user. For example, the user can access and track their emotional state by dragging an icon along a scale from 0 percent to 100 percent. Each update is timestamped, creating a detailed log of fluctuations throughout the day, allowing user to visualise changes in their overall wellbeing. Additionally, the user can log their mood and emotion data using a colour coded key. In some embodiments, the user device 104, based on instructions from the server 106, is configured to provide bonus points to the user when the user logs his mood and emotion data over a set period. In some embodiments, the user input data includes physical data of the user, for example, temperature, heart rate, breathing rate, and other similar data captured by the user device 104 to determine the mood and emotion data of the user.

Additionally, or alternatively, in some embodiments, the user input data corresponds to a user generated input that provides implicit data regarding the emotional state of the user. The user generated inputs include direct user inputs, for example, via journals or reflective prompts. The journaling feature provided in the web resource 118 provides the user both freeform and prompt-based journaling options. The prompt-based journaling is themed around the specific character or theme, emotional states, or physical product, guiding the users to reflect on specific aspects of their wellness journey. Alternatively, freeform journals facilitate unstructured writing on any topic. In some embodiments, the web resource 118 includes a notes section in which the user can log thoughts, events, or reflections about the day.

At 410, the server 106 receives the user input data associated with the mental health of the user via the server transceiver 140. At 412, the server 106 determines the mental health state of the user based on the user input data. In accordance with various embodiments, when the user input data corresponds to the mood and emotion data, the mental health state corresponds to the emotional state of the user. When the user input data corresponds to the user generated input, the server processor 146 determines the mental health state of the user by analysing the implicit data provided in the user generated inputs to identify the one or more indicators using the machine learning models 152. The server processor 146 then determines the mental health state of the user based on the one or more indicators using the machine learning models 152. In accordance with various embodiments, the one or more indicators correspond to one or more of sentiments, linguistic features, topics, behavioural shift, usage of predefined phrases or words, thought patterns, and reasoning derived from the implicit data provided in the user generated inputs. For example, the server processor 146 determines the mental health state of the user by analysing journal entries whether typed or scanned from handwritten pages and/or data entered in the notes section for recurring themes, emotional patterns, and keywords using the machine learning model 152.

At 414, the server processor 146 determines the digital mental health resources corresponding to the mental health state of the user using one or more machine learning models 152. For example, when the mood and emotion data indicates that the emotional state of the user is anxious, and the same emotional state is inputted by the user three times in a week, the machine learning models 152, when executed by the machine learning module 154, prioritizes suggesting resources related to managing anxiety, such as breathing exercises or relaxation techniques. In an exemplary embodiment, the machine learning models 152 use the text entries in the journal as data points for understanding emotional trends, language use, and patterns that indicates the mental health state of the user. For example, when the user's journal consistently reflects themes of stress during exams, the machine learning model 152 identifies the mental health state of the user as ‘stressed’ and proactively recommends stress-management strategies or resources during similar timeframes.

At 416, the server 106 provides the digital mental health resources to the user device 104 through the user device interface 122. In some embodiments, the one or more recommendations are delivered in various forms, such as text notifications, guided audio sessions, or links to mental health resources.

At 418, the server 106 monitors interactions of the user with the web resource 118 and the digital mental health resources provided through the user device interface 122 to generate the behavioural data and the resource usage data, respectively. The behavioural data corresponds to user interaction data with the web resource 118 on the user device 104. The behavioural data includes one or more of a frequency and a time of activation of the tag 102, a frequency and a time of entry of the user input data, and a frequency and a time of accessing one or more predefined resources provided on the web interface 118. For example, the behavioural data captures the user engagement with different features of the web resource 118, such as, a frequency of activation/tapping of the tag 102, a frequency of entry of mood and emotion data, a frequency of accessing a personalized ‘safety plan’ resource provided in the web resource 118 that supports the user experiencing panic attack, suicidal thoughts, or self-harm urges, and a frequency of entering data points via journals or reflective prompts.

The resource usage data corresponds to interaction data of the user with the digital mental health resources. The resource usage data includes data around how often and which type of digital mental health resources (for example, meditation, crisis resources, grounding exercises) the user engages with. In accordance with various embodiments, the user device 104 captures the behavioural data and the resource usage data associated with the interaction of the user with the web resource 118 and the digital mental health resources selected by the user, and transmits it to the server 106. For example, the data associated with the digital mental health resources selected by the user includes data associated with a use (for example, a frequency and a type) of a mental health resource (for example, meditation, crisis resources, grounding exercises) by the user.

At 420, the server processor 146 provides the user input data, the behavioural data, and the resource usage data to the machine learning models 152 to identify unlabelled clusters, patterns, or relationships in the user input data, the behavioural data, and the resource usage data associated with the user. At 422, the server processor 146 dynamically updates the digital mental health resources provided to the user through the user device interface 122 based on the identified clusters, patterns, or relationships using the machine learning models 152. For example, the machine learning models 152 determine that the user who regularly tracks feelings of stress in the morning also tend to benefit from a particular morning routine. Accordingly, the machine learning models 152 also provide suggestion even when the user hasn't explicitly requested for a suggestion. Such personalized approach allows the machine learning models 152 to provide updated and more tailored mental health resources over time, based on the identified clusters, patterns, or relationships.

The machine learning models 152 recommend tailored digital mental health resources based on the behavioural data and the resource usage data. In accordance with various embodiments, the server processor 146, upon updating the digital mental health resources, also revises the web resource 118 associated with the tag 102. For example, when the user engages with the safety plan or crisis resources, the machine learning model 152 provides follow up support through guided check-ins, offering options for crisis intervention services or coping strategies. In case there is a change in usage of web resource 118, the machine learning model 152 initiates an interactive check-in, or conducts a conversation with the user that leads to digital mental health resource recommendations to the user.

For example, when the behavioural data indicates that the user has accessed the ‘safety plan’ resource during stressful times, the machine learning models 152 recommend follow-up actions such as, encouraging the user to check in with a mental health professional or using a grounding technique. Similarly, when the resource usage data indicates that the user frequently uses meditation resources for relaxation, the machine learning models 152 suggest meditation exercises during similar emotional states or stress levels to improve relevance and effectiveness.

In accordance with various embodiments, the safety plan corresponds to a crisis management tool. The server processor 146 is configured to guide the user through an interactive process to create a personalised safety plan, which includes crisis resources (for example, hotlines, chat services), safety contacts and local support options. The safety plan provides one click support, enabling the user to send automated texts to designated safety contacts, sharing their current state and specifying the type of help needed (for example, a phone call or a text). In case the safety plan is activated by the user on the user device 104, the machine learning module 154 monitors the user's engagement and follows up in timely manner with the user (for example, 1 hour later, the next day).

At 424, the server processor 146 obtains the user feedback on the digital mental health resources provided to the user through the user device interface 122 and trains the machine learning models 152 based on the user feedback using the reinforcement learning technique at 426. At 428, the server processor 146 dynamically updates the digital mental health resources provided to the user through the user device interface 122 based on the user feedback using the machine learning models 152. The server 106 utilizes the user feedback to refine and optimize the recommendations over time, making them more personalised and effective. For example, when the user responds positively to journaling as a coping tool, the machine learning model 152 suggests more journaling prompts in the recommendations. In accordance with various embodiments, the server processor 146, upon updating the digital mental health resources, also revises the web resource 118 associated with the tag 102.

At 430, the server processor 146 continuously monitors the web resource 118 to obtain updated data associated with one or more of the user input data, the behavioural data, the resource usage data, and the user feedback. In accordance with various embodiments, the server processor 146 repeats the step associated with updating the digital mental health resources using the machine learning models 152 upon receiving the updated data. At 432, the server processor 146 determines whether the updated data associated with the user input data is received. When the server processor 146 determines that the updated data associated with the user input data is received, the method loops back to 412. If not, the method proceeds to 434. At 434, the server processor 146 determines whether the updated data associated with the behavioural data and/or the resource usage data is received. When the server processor 146 determines that the updated data associated with the behavioural data and/or the resource usage data is received, the method loops back to 420. If not, the method proceeds to 436. At 436, the server processor 146 determines whether the updated data associated with the user feedback is received. When the server processor 146 determines that the updated data associated with the user feedback is received, the method loops back to 426. If not, the method loops back to 406 and renders a revised web resource 118 with the updated digital mental health resources on the user device interface 122.

In accordance with various embodiments, by continuously monitoring the web resource 118 to obtain updated data, the server processor 146 is configured to revise the web resource 118 associated with the URL when the digital mental health resources are updated. In accordance with various embodiments, when, in a sequential instance occurring successively to or after an initial instance, the user device 104 is positioned within the predefined distance from the tag 102, the user device 104 is configured to render, on the user device interface 122, the revised web resource 118 corresponding to the URL that enables the server transceiver 140 to provide the updated digital mental health resources to the user through the user device interface 122. In some embodiments, when the tag stores multiple URLs corresponding to multiple web resources, the server processor 146 revises each web resource of the multiple web resources to reflect the updated digital mental health resources based on the updated data. The server processor 146 continuously keeps revising the web resource 118 corresponding to the URL with the updated digital mental health resources. In some embodiments, the server processor 146 also generates reports that summaries user engagement and behaviour and provides it to the user device 104 for display to the user. These reports are generated based on the user data and highlight trends, such as the number of tag activations, the prevalence of specific emotions (for example, anxiety or stress), and the coping tool usage frequency.

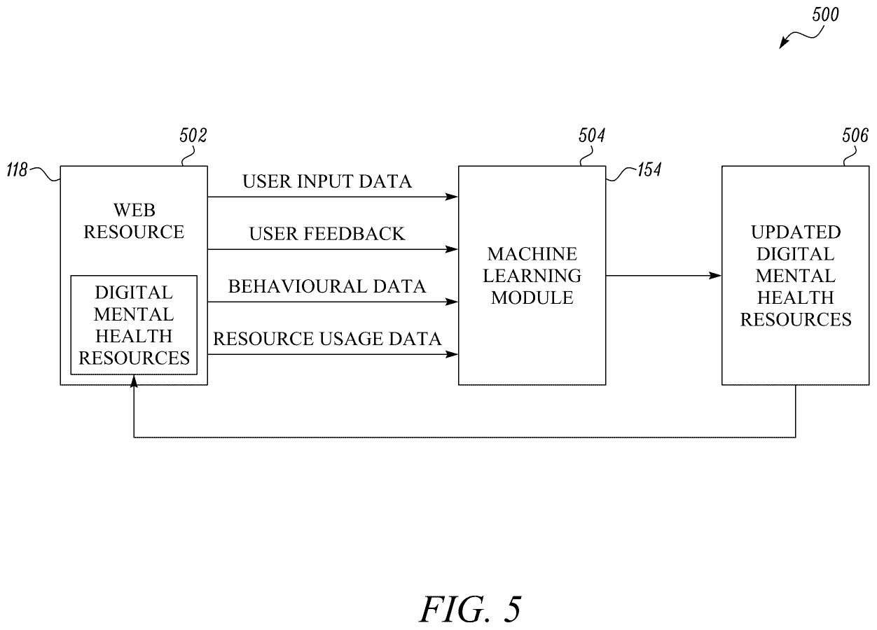

FIG. 5 illustrates an optimization cycle 500 for optimizing the digital mental health resources provided by the server 106. At 502, the web resource 118 with the digital mental health resources is rendered on the user device 104 to obtain the user input data, the behavioural data, the resource usage data, and the user feedback from the user of the user device 104. At 504, the user input data, the behavioural data, the resource usage data, and the user feedback is received by the machine learning module 154 of the server 106. At 506, the machine learning module 154 of the server 106 determines the updated digital mental health resources based on the user input data, the behavioural data, the resource usage data, and the user feedback. The updated digital mental health resources are then utilized to determine the revised web resource 118 including the updated digital mental health resources. Subsequently, when the user device 104 receives the URL from the tag 102, the method loops back to 502 to render the revised web resource 118 including the updated digital mental health resource on the user device interface 122 of the user device 104.

In some embodiments, each object 112, 116 is associated with a specific resource library 156. By purchasing these objects 112, 116, the user can unlock exclusive content and resources linked to a specific character or theme. The server 106 features profiles for each character, which represent different emotional states. The profiles include the characters image, a selection of their preferred mental wellness tools, and a feed of social media like post. Each character profile includes a comment wall for an in-universe interaction, enhancing the immersive experience. Further, when the user purchases products associated with the character, they can add that character to their in-application friend list. This provides access to animations and special interactions from the character, enriching the user engagement through an influencer style content and a personalised social environment. Moreover, the users can add and interact with real-life friends by connecting via phone numbers using the friend list function provided on the web resource 118. Once added, users can communicate with each other and share supportive messages or updates.

In some embodiments, the system 100 is implemented to make mental wellness practices easily accessible for Business-to-Business (B2B) and/or Business-to-Consumer (B2C) customers. For example, the system 100 is implemented for an employer that includes an employer portal which serves as an interface for employees for promoting workplace wellness. Additionally, the system 100 is implemented for a school that includes a school administrator portal which serves as an interface for an educator and a school staff to access anonymized or non-anonymized information and insights about a student or the user engagement with mental wellness program. The portal provides features such as privacy and consent, safety and support, productivity and wellness challenges, mental health resource library 156, anonymous communication, and onboarding and customization. The portal also provides aggregated metrics, and anonymous data on the engagement trends, such as, stress management exercises and wellness challenges without providing access to personal data of the individuals.

In the hereinbefore specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. The benefits, advantages, solutions to problems, and any element(s) that can cause any benefit, advantage, or solution to occur or become more pronounced are not to be construed as a critical, required, or essential feature or elements of any or all the claims. The invention is defined solely by the appended claims including any amendments made during the pendency of this application and all equivalents of those claims as issued.

We claim

1. A system for remotely managing mental health of a user, the system comprising:
an object;
a tag adhered to the object, the tag including a memory for storing a uniform resource locator (URL) associated with a web resource for providing digital mental health resources;
a user device configured to:
receive the URL from the tag when the user device is positioned within a predefined distance from the tag,
render, on a user device interface of the user device, the web resource corresponding to the URL, and
obtain, via the user device interface, user input data associated with the mental health of the user through one or more graphical elements of the web resource; and
a server associated with the web resource and communicatively coupled to the user device, the server configured to:
receive, by a server transceiver, the user input data associated with the mental health of the user,
determine, by a server processor, mental health state of the user based on the user input data,
determine, by the server processor, digital mental health resources corresponding to the mental health state of the user using one or more machine learning models,
provide, by the server transceiver, the digital mental health resources to the user through the user device interface,
monitor, by the server processor, interactions of the user with the web resource and the digital mental health resources provided through the user device interface to generate behavioural data and resource usage data, respectively,
provide, by the server processor, the user input data, the behavioural data, and the resource usage data to the one or more machine learning models to identify unlabelled clusters, patterns, or relationships in the user input data, the behavioural data, and the resource usage data associated with the user,
dynamically update, by the server processor, the digital mental health resources provided to the user through the user device interface based on the identified clusters, patterns, or relationships using the one or more machine learning models,
obtain, by the server processor, user feedback on the digital mental health resources provided to the user through the user device interface,
train, by the server processor, the one or more machine learning models based on the user feedback using a reinforcement learning technique,
dynamically update, by the server processor, the digital mental health resources provided to the user through the user device interface based on the user feedback using the one or more machine learning models,
continuously monitor, by the server processor, the web resource to obtain updated data associated with one or more of the user input data, the behavioural data, the resource usage data, and the user feedback, and
repeat, by the server processor, the step associated with updating the digital mental health resources using the one or more machine learning models upon receiving the updated data,
wherein the user device is further configured to:
display, on the user device interface, the digital mental health resources provided by the server processor.

2. The system of claim 1, wherein the server processor is further configured to:
train the one or more machine learning models by:
obtaining a first training dataset including historical data of indicators associated with a plurality of mental health states and corresponding digital mental health resources to manage each mental health state of the plurality of health states;
training, using a supervised learning technique, the one or more machine learning models on the first training dataset to (i) define one or more indicators corresponding to each mental health state of the plurality of health states, (ii) identify the mental health state of a user based on the one or more indicators, and (iii) determine digital mental health resources corresponding to each mental health state of the plurality of health states;
obtaining a second training dataset including historical user input data, historical behavioural data, and historical resource usage data of a plurality of users;
training, using an unsupervised learning technique, the one or more machine learning models on the second training dataset to identify unlabelled clusters, patterns, or relationships in the user input data, the behavioural data, and the resource usage data of the plurality of users.

3. The system of claim 2, wherein the user input data corresponds to mood and emotion data explicitly defining an emotional state of the user, a user generated input providing implicit data regarding the emotional state of the user, or a combination thereof.

4. The system of claim 3, wherein when the user input data corresponds to mood and emotion data, the mental health state corresponds to the emotional state of the user.

5. The system of claim 3, wherein when the user input data corresponds to the user generated input, the server processor is configured to determine the mental health state of the user by:
analysing, using the one or more machine learning models, the implicit data provided in the user generated inputs to identify the one or more indicators; and
determining, using the one or more machine learning models, the mental health state of the user based on the one or more indicators,
wherein the one or more indicators correspond to one or more of sentiments, linguistic features, topics, behavioural shift, usage of predefined phrases or words, thought patterns, and reasoning derived from the implicit data provided in the user generated inputs.

6. The system of claim 5, wherein the user generated input corresponds to journal entries provided by the user.

7. The system of claim 1, wherein by continuously monitoring the web resource to obtain updated data, the server processor is configured to:
revise the web resource associated with the URL when the digital mental health resources are updated, and
when, in a sequential instance occurring successively to or after an initial instance, the user device is positioned within the predefined distance from the tag, the user device is configured to render, on the user device interface, the revised web resource corresponding to the URL that enables the server transceiver to provide updated digital mental health resources to the user through the user device interface.

8. The system of claim 1, wherein the tag stores multiple URLs corresponding to multiple web resources, wherein the server processor is configured to update each web resource of the multiple web resources based on the updated data.

9. The system of claim 1, further comprising:
a database configured to store the user input data, the behavioural data, the resource usage data, the user feedback, and the digital mental health resources.

10. The system of claim 1, wherein the tag is a near field communication (NFC) tag and further wherein the user device is configured to activate the NFC tag when the user device is positioned within the predefined distance from the NFC tag to receive the URL from the NFC tag.

11. The system of claim 1, wherein the web resource corresponds to a web portal or a web page, and further wherein the web resource is accessed through a web browser on the user device or within a mobile application installed on the user device.

12. The system of claim 1, wherein the digital mental health resources include one or more of videos, websites, documents, books, podcasts, exercises, meditation support, access to support groups, monthly reports, interactive coping activities, chat support, challenges, or alerts.

13. The system of claim 1, wherein identifying unlabelled clusters, patterns, or relationships in the user input data, the behavioural data, and the resource usage data associated with the user includes identifying connections between different mental health states of the user.

14. The system of claim 1, wherein the behavioural data includes one or more of a frequency and a time of activation of the tag, a frequency and a time of entry of the user input data, and a frequency and a time of accessing one or more predefined resources provided on the web interface.

15. A method for remotely managing mental health of a user, the method comprising:
receiving, by a user device transceiver of a user device, a uniform resource locator (URL) from a tag when the user device is positioned within a predefined distance from the tag, wherein the URL is associated with a web resource for providing digital mental health resources;
rendering, on a user device interface of the user device, the web resource corresponding to the URL;
obtaining, via the user device interface, user input data associated with the mental health of the user through one or more graphical elements of the web resource;
receiving, by a server transceiver of a server, the user input data associated with the mental health of the user;
determining, by a server processor of the server, mental health state of the user based on the user input data;
determining, by the server processor, digital mental health resources corresponding to the mental health state of the user using one or more machine learning models;
providing, by the server transceiver, the digital mental health resources to the user through the user device interface of the user device;
monitoring, by the server processor, interactions of the user with the web resource and the digital mental health resources provided through the user device interface of the user device to generate behavioural data and resource usage data, respectively;
providing, by the server processor, the user input data, the behavioural data, and the resource usage data to the one or more machine learning models to identify unlabelled clusters, patterns, or relationships in the user input data, the behavioural data, and the resource usage data associated with the user;
dynamically updating, by the server processor, the digital mental health resources provided to the user through the user device interface based on the identified clusters, patterns, or relationships using the one or more machine learning models;
obtaining, by the server processor, user feedback on the digital mental health resources provided to the user through the user device interface;
training, by the server processor, the one or more machine learning models based on the user feedback using a reinforcement learning technique;
dynamically updating, by the server processor, the digital mental health resources provided to the user through the user device interface based on the user feedback using the one or more machine learning models;
continuously monitoring, by the server processor, the web resource to obtain updated data associated with one or more of the user input data, the behavioural data, the resource usage data, and the user feedback;
repeating, by the server processor, the step associated with updating the digital mental health resources using the one or more machine learning models upon receiving the updated data; and
displaying, on the user device interface, the digital mental health resources provided by the server processor.

16. The method of claim 15, further comprising:
training, by the server processor, the one or more machine learning models by:
obtaining a first training dataset including historical data of indicators associated with a plurality of mental health states and corresponding digital mental health resources to manage each mental health state of the plurality of health states;
training, using a supervised learning technique, the one or more machine learning models on the first training dataset to (i) define one or more indicators corresponding to each mental health state of the plurality of health states, (ii) identify the mental health state of a user based on the one or more indicators, and (iii) determine digital mental health resources corresponding to each mental health state of the plurality of health states;
obtaining a second training dataset including historical user input data, historical behavioural data, and historical resource usage data of a plurality of users;
training, using an unsupervised learning technique, the one or more machine learning models on the second training dataset to identify unlabelled clusters, patterns, or relationships in the user input data, the behavioural data, and the resource usage data of the plurality of users.

17. The method of claim 16, wherein the user input data corresponds to mood and emotion data explicitly defining an emotional state of the user, a user generated input providing implicit data regarding the emotional state of the user, or a combination thereof.

18. The method of claim 17, wherein when the user input data corresponds to mood and emotion data, the mental health state corresponds to the emotional state of the user.

19. The method of claim 17, wherein when the user input data corresponds to the user generated input, determining the mental health state of the user comprises:
analysing, by the server processor, the implicit data provided in the user generated inputs to identify the one or more indicators using the one or more machine learning models; and
determining, by the server processor, the mental health state of the user based on the one or more indicators using the one or more machine learning models,
wherein the one or more indicators correspond to one or more of sentiments, linguistic features, topics, behavioural shift, usage of predefined phrases or words, thought patterns, and reasoning derived from the implicit data provided in the user generated inputs.

20. The method of claim 15, wherein the tag is a near field communication (NFC) tag and further wherein the method comprises:
activating the NFC tag when the user device is positioned within the predefined distance from the NFC tag to receive the URL from the NFC tag.
